(19) United States
(12) Patent Application Publication
(10) Pub. No.: US 2016/0166433 A1
(43) Pub. Date: Jun. 16, 2016

(54) SURGICAL INSTRUMENT

(71) Applicant: Carl Zeiss Meditec AG, Jena (DE)

(72) Inventor: Stefan CZAJA, Glienicke (DE)

(21) Appl. No.: 14/965,202

(22) Filed: Dec. 10, 2015

(30) Foreign Application Priority Data
Dec. 12, 2014 (DE) 10 2014 118 575.7

(51) Int. Cl. A61F 9/007; A61B 1/07; A61B 17/3201; A61B 17/34; A61B 17/28

(52) U.S. Cl. CPC A61F 9/00763; A61B 17/3421; A61B 17/28; A61B 17/3201; A61B 1/07; A61B 2217/005

(57) ABSTRACT

A surgical instrument (101) is made available, in particular a microsurgical instrument, with a rod-shaped or sleeve-shaped portion (103) that comprises a proximal end (113) and a distal end (111). The rod-shaped or sleeve-shaped portion (103) is made of a transparent ceramic and has, at its proximal end (113), an attachment piece (117) for an optical fiber output (119) which allows light emerging from the optical fiber output (119) to be introduced into the transparent ceramic. The transparent ceramic of the rod-shaped or sleeve-shaped portion (103) is provided with a non-transparent coating (123), except in a light outlet area (125).

The present invention relates to a surgical instrument and in particular to a microsurgical instrument, for example a microsurgical instrument for vitrectomy.

In minimally invasive surgical interventions, and in particular in microsurgical interventions in the human body, it is not generally possible to work with external illumination of the operating site. Instead, endoillumination, i.e. illumination from within the body, is necessary. For endoillumination, an additional opening is often created through which, for example, a light pipe is introduced into the inside of the body. In vitrectomy for example, in which the vitreous body filling the space between the posterior wall of the eyeball and the crystalline lens is removed from the eye, three openings are created in the eyeball by means of trocars. Infusion of a liquid takes place through the first opening, said liquid replacing the vitreous body, as the latter is removed, in order to prevent a collapse of the eyeball during the intervention. A light pipe is inserted into the eye through the second opening in order to illuminate the interior of the eyeball. The third opening serves for the insertion of treatment instruments with which the surgeon breaks up the vitreous body and removes it by suction. Instruments for breaking up the vitreous body and removing it by suction are described, for example, in U.S. Pat. No. 8,187,293 B2 and U.S. Pat. No. 8,845,666 B2.

During a vitrectomy procedure, the operating surgeon not only holds the actual vitrectomy instrument itself, but also the light pipe in order to obtain suitable illumination in the area surrounding the tip of the vitrectomy instrument. It would be advantageous, however, if the surgeon did not have to concern himself with the positioning of the light pipe. He would then have both hands free for treatment instruments, so that he could perform the procedure with two hands. Similar problems can also arise in other microsurgical interventions, for example in vascular surgery or neurosurgery.

EP 0 566 359 A1 and DE 43 06 553 A1 disclose trocars comprising cannulas into which optical fibers extending as far as the distal end of the cannula are integrated. By means of the optical fibers, illumination light can be routed to the distal end of the cannula, such that illumination of the operating site is possible via the cannula of the trocars. However, the cannulas of such trocars have a relatively complex design, which makes the production of the trocars more complicated and therefore more expensive. In addition, the integration of optical fibers into a trocar cannula requires a corresponding installation space within the cannula, such that this configuration is unsuitable in particular for microsurgical trocars.

The object of the present invention is therefore to make available a surgical instrument and in particular a microsurgical instrument which helps overcome the described problems.

This object is achieved by a surgical instrument, in particular a microsurgical instrument, according to claim 1. The dependent claims contain advantageous embodiments of the invention.

A surgical instrument according to the invention, which can in particular be a microsurgical instrument, for example for vitrectomy, comprises a rod-shaped or sleeve-shaped portion which has a proximal end and a distal end and which is made of a transparent ceramic. At the proximal end of the rod-shaped or sleeve-shaped portion, an attachment piece is present for an optical fiber output, said attachment piece allowing light emerging from the optical fiber output to be introduced into the transparent ceramic. The transparent ceramic of the rod-shaped or sleeve-shaped portion is provided with a non-transparent coating, for example a varnish, a spray coating or the like, except in a light outlet area.

By virtue of the fact that the rod-shaped or sleeve-shaped portion of the surgical instrument is made of a transparent ceramic, the light introduced at the proximal end into the ceramic can reach the light outlet area and can there emerge from the rod-shaped or sleeve-shaped portion. In the other portions, the non-transparent coating prevents light from emerging from the transparent ceramic, and it is thus possible to avoid undesired light output that could cause reflection interference.

A polycrystalline ceramic can be used in particular as the transparent ceramic, for example aluminum oxynitride, which is also known under the trade name Alon, or aluminum oxide, which is also known as corundum and sapphire crystal.

By virtue of its hardness, the transparent ceramic is suitable in particular for the production of stable rod-shaped or sleeve-shaped portions with small and very small dimensions, in particular with small and very small diameters. A surgical instrument according to the invention is therefore highly suitable as a microsurgical instrument, for example for vascular surgery, neurosurgery or, in particular, for ophthalmic surgery.

The light outlet area of the rod-shaped or sleeve-shaped portion preferably lies closer to the distal end of the rod-shaped or sleeve-shaped portion than to the proximal end of the rod-shaped or sleeve-shaped portion, wherein the light outlet area can in particular be arranged directly at the distal end of the rod-shaped or sleeve-shaped portion. Since the operating site is located close to the distal end of the rod-shaped or sleeve-shaped portion during the intervention, the illumination can in this way be provided close to the operating site. Irrespective of the arrangement of the light outlet area in the rod-shaped or sleeve-shaped portion, said light outlet area can have a shape that allows the light to emerge in a desired light outlet direction and/or with a desired radiation characteristic. This can be achieved, for example, by a suitable ground surface of the light outlet area.

The present invention allows surgical instruments to be used at the same time as endoilluminators for illuminating the operating site. Since the actual operating instrument is arranged at the distal end of the rod-shaped or sleeve-shaped portion, the illumination afforded by the surgical instrument according to the invention is always provided at the site of the actual manipulation or at least in very close proximity to this site.

In a first particular embodiment, the surgical instrument according to the invention can be designed as a vitrector with a tubular cutter, wherein the cutter then comprises the rod-shaped or sleeve-shaped portion made of a transparent ceramic. A vitrector is used, during a vitrectomy procedure, to remove vitreous substance in small pieces from the eye. The distal end of the cutter breaks up vitreous substance into small pieces, which are then aspirated through the cutter. By virtue of the fact that the cutter comprises a rod-shaped or sleeve-shaped portion made of a transparent ceramic, the site at which vitreous substance is broken up and removed from the eye can be illuminated with the vitrector right through the cutter. Since the surgeon does not then need to concern himself with the positioning of a light pipe, he has his second hand free and can, for example, work with two vitrectors simultaneously using both hands, as a result of which the period of time for performing a vitrectomy procedure can be shortened.

In a second particular embodiment, the surgical instrument can be designed as a trocar with a cannula, wherein the cannula forms the rod-shaped or sleeve-shaped portion made of a transparent ceramic. A trocar is inserted into an opening that has been created artificially in the body for a surgical intervention, such that the distal end of its cannula is located inside the body and serves to ensure that the opening in the body is kept open during the intervention. The surgical treatment instruments needed for the intervention are then guided through the trocar cannula to the site of the intervention inside the body. The distal end of the trocar cannula arranged inside the body is located near the treatment site and/or is directed toward the treatment site, such that the treatment site can be illuminated through the trocar cannula. It is therefore possible to dispense with a further artificially created opening in the body for insertion of a means for illuminating the treatment site, without space in the trocar cannula being used for the passage of, for example, a lighting rod or a lighting fiber.

In a third particular embodiment, the surgical instrument can be designed as micro forceps in which two movable grip portions spaced apart from each other by a spring force are located at the end of a rod guided through a tube. The rod is movable in the longitudinal direction of the tube between a position in which the grip portions are located outside the tube and spaced apart from each other and a position in which the grip portions are located partially in the tube and are pressed together by a wall portion of the tube counter to the spring force. In this particular embodiment, the tube forms the rod-shaped or sleeve-shaped portion made of a transparent ceramic. Since the micro forceps allow the site at which a manipulation takes place with the micro forceps to be illuminated through the tube, it is possible to dispense with an additional lighting rod or an additional lighting fiber, such that a surgeon using the micro forceps has his second hand free for a further treatment instrument. In this way, for example, the gripping with the forceps can be assisted by a spatula, with which a structure to be gripped can be lifted.

In a fourth particular embodiment, the surgical instrument can be designed as micro scissors in which two movable blades spaced apart from each other by a spring force are located at the end of a rod guided through a tube. The rod is movable in the longitudinal direction of the tube between a position in which the blades are located outside the tube and spaced apart from each other and a position in which the blades are located partially in the tube and are pressed together by a wall portion of the tube counter to the spring force. As in the third particular embodiment, the tube in the fourth particular embodiment forms the rod-shaped or sleeve-shaped portion made of a transparent ceramic. Since the micro scissors allow the site at which cutting takes place with the micro scissors to be illuminated through the tube, it is possible to dispense with an additional lighting rod or an additional lighting fiber, such that a surgeon using the micro scissors has his second hand free for a further treatment instrument. In this way, for example, the tissue that is to be cut can be held with micro forceps.

In a fifth particular embodiment, the surgical instrument can be designed as a suction instrument (a so-called backflush instrument). Such an instrument comprises a suction tube, which forms the rod-shaped or sleeve-shaped portion made of a transparent ceramic. Since the suction instrument allows the site at which suction takes place to be illuminated through the suction tube, it is possible to dispense with an additional lighting rod or an additional lighting fiber, such that a surgeon using the suction instrument has his second hand free for a further treatment instrument. In the case of a retinal detachment, for example, in which liquid has accumulated under the detached area of the retina, this makes it possible to lift the retina with micro forceps, after which the liquid located underneath is aspirated through the opening via which the liquid has arrived under the retina.

Further features, properties and advantages of the present invention will become clear from the following description of illustrative embodiments and by reference to the attached figures.

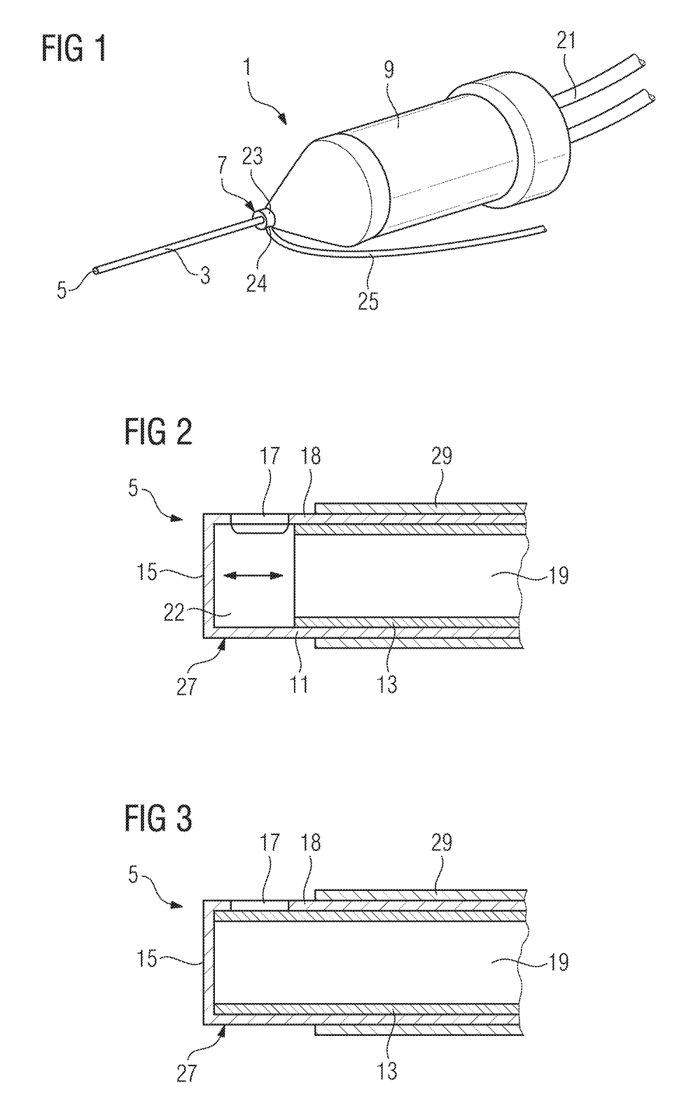

FIG. 1 shows a vitrector as a first illustrative embodiment of a surgical instrument according to the invention.

FIG. 2 shows a cutter of the kind that can be used in the vitrector according to FIG. 1, in a first cutter position.

FIG. 3 shows the cutter from FIG. 2 in a second cutter position.

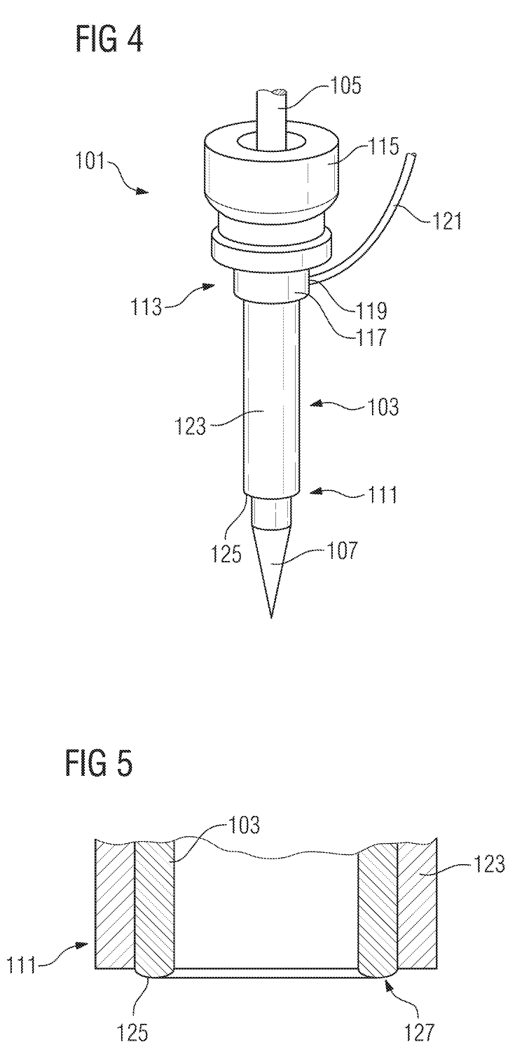

FIG. 4 shows a trocar with a cannula and with a lancet guided in the cannula, as a second illustrative embodiment of a surgical instrument according to the invention.

FIG. 5 shows the distal end of the cannula of the trocar.

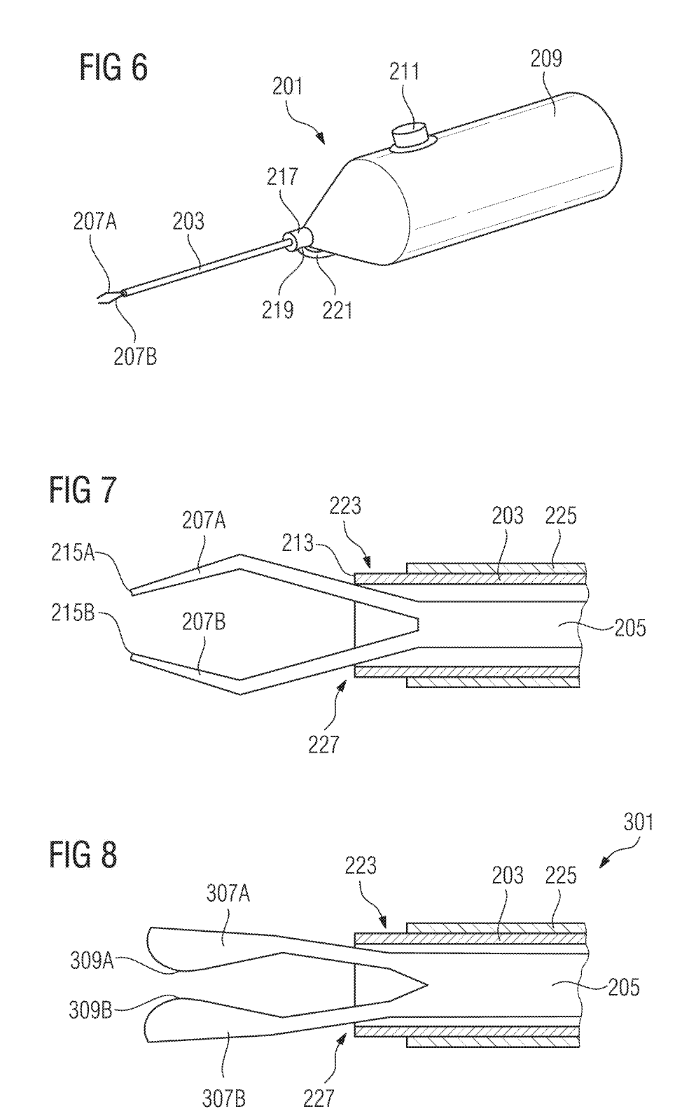

FIG. 6 shows micro forceps as a third illustrative embodiment of a surgical instrument according to the invention.

FIG. 7 shows the distal end of the forceps.

FIG. 8 shows the distal end of micro scissors, which represent a fourth illustrative embodiment of a surgical instrument according to the invention.

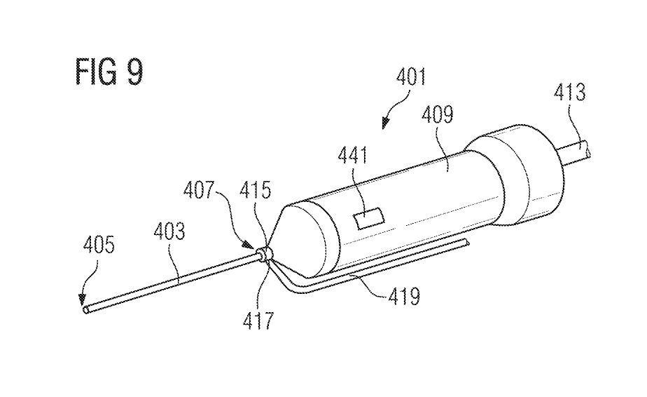

FIG. 9 shows a suction instrument, which represents a fifth illustrative embodiment of a surgical instrument according to the invention.

Illustrative embodiments of surgical instruments according to the invention are described below with reference to the figures. A common aspect of all these surgical instruments is that they have a rod-shaped or sleeve-shaped portion which is made of a transparent ceramic and, at its proximal end, has an attachment piece for an optical fiber output. By way of this attachment piece, light emerging from the optical fiber output can be introduced into the transparent ceramic. Except for a light outlet area, the rod-shaped or sleeve-shaped portion is provided with a non-transparent coating, such that the light can emerge from the transparent ceramic only at a desired location. The coating can be, for example, a varnish, a spray coating, or a coating applied by chemical vapor deposition (CVD) or physical vapor deposition (PVD) from the gas phase.

In all of the illustrative embodiments, the transparent ceramic used can be a transparent polycrystalline ceramic, for example aluminum oxide, also known as corundum or sapphire crystal, and in particular aluminum oxynitride, also known under the trade name Alon. However, other transparent ceramics can in principle also be used.

FIG. 1 shows a vitrector 1 as a first illustrative embodiment of a surgical instrument according to the invention. This vitrector 1 comprises a cutter 3 with a distal end 5 and a proximal end 7. The proximal end 7 is adjoined by a handpiece 9, in which the drive of the cutter is arranged.

The cutter comprises, as its main components, an outer tube 11 and an inner tube 13 which is arranged movably in the interior of the outer tube 11 along the longitudinal axis thereof (cf. FIGS. 2 and 3). The outer tube 11 is closed at the distal end 5 of the cutter by a front wall 15 and, in the area of the distal end 5 of the cutter, has an opening 17 in its circumferential wall 18.

The inner tube 13 is open at its distal end 14 and has a lumen 19 which, in the area of the handpiece 9, is connected to a suction device, of which FIG. 1 shows only a suction hose 21 leading out from the handpiece 9. The inner tube 13 is moreover connected to an actuator, which is arranged in the interior of the handpiece 9 and which allows the inner tube 13 to be moved to and fro inside the outer tube 11 along the common longitudinal axis, as is indicated by a double arrow in FIG. 2. By moving to and fro, the inner tube 13 can be brought to a first position with respect to the outer tube 11, in which position it completely frees the opening 17 in the circumferential wall 18 of the outer tube 11. This position of the inner tube 13 is shown in FIG. 2. Moreover, by means of moving to and fro with respect to the outer tube 11, the inner tube 13 can be brought to a second position, in which it completely closes the opening 17 in the circumferential wall 18 of the outer tube 11, as is shown in FIG. 3. The external diameter of the inner tube 13 is chosen such that only a minimal circumferential gap remains between the outer surface of the inner tube 13 and the inner surface of the outer tube 11, said circumferential gap being exactly of such a size as to permit the movement to and fro of the inner tube 13.

During the operation of the cutter, the inner tube 13 moves rapidly to and fro at a rate of typically 20 Hz or more. When, during this reciprocating motion, the inner tube 13 is located, with respect to the outer tube 11, in the position shown in FIG. 2, the suction device is used to aspirate vitreous substance through the opening 17 in the circumferential wall 18 of the outer tube 11 into the space 22 formed between the front wall 15 of the outer tube 11 and the open distal end 14 of the inner tube 13. During the movement of the inner tube 13 to the position shown in FIG. 3, the vitreous substance aspirated into the space 22 is separated from the rest of the vitreous substance and aspirated through the lumen 19 of the inner tube 13. In this way, with the aid of the cutter, the vitreous substance in the eye is broken up and removed.

In the present illustrative embodiment, the outer tube 11 is made of aluminum oxynitride, which is transparent to visible light. At the proximal end 7 of the cutter, the outer tube 11 is provided with an attachment piece for the outlet end 24 of an optical fiber 25. Illumination light originating from a remote light source is conveyed by the optical fiber 25 to the attachment piece 23, where the illumination light emerging from the outlet end 24 is coupled into the transparent ceramic of the outer tube 11. Through the transparent ceramic of the outer tube 11, the coupled-in light then reaches a light outlet area 27 of the outer tube 11, which light outlet area 27 is arranged at the distal end 5 of the cutter. Outside the attachment piece 23, the outer surface of the outer tube 11, except for the light outlet area 27, is provided with a coating 29 which is non-transparent to visible light and which ensures that the light coupled into the transparent material of the outer tube 11 does not emerge outside the light outlet area 27.

The described vitrector 1 allows illumination light to be conveyed, with the aid of the cutter, directly to the site at which the removal of the vitreous substance actually takes place. In other words, the cutter 3 provides for its own illumination within its working area. It is therefore possible to dispense with additional illumination by means of a light pipe. In this way, the surgeon has his other hand free, thus allowing both hands to be used to perform the intervention.

FIGS. 4 and 5 show a second illustrative embodiment of a surgical instrument according to the invention. FIG. 4 shows, as the second illustrative embodiment, a trocar 101 which comprises a cannula 103 and a lancet 105 with a tip 107, while FIG. 5 shows the distal end 111 of the trocar cannula 103. The lancet 105, which is guided through the interior of the cannula 103 and protrudes from the cannula 103, serves to puncture tissue, for example to puncture the eyeball, during the insertion of the trocar 101. After the puncturing, the lancet 105 is removed and the cannula 103 remains in the punctured tissue, in order to keep open an access to the interior of the tissue.

The cannula 103 has an open distal end 111, which is located inside the body after the trocar has been fitted in place, and a proximal end 113, which is located outside the body and to which an instrument port 115 is connected. After the lancet 105 has been withdrawn, treatment instruments can be inserted through the instrument port 115 and through the trocar cannula 103 into the inside of the body. If the trocar 101 shown in FIG. 4 is a microsurgical trocar for vitrectomy, it is possible, for example, for the cutter of the vitrector shown in FIG. 1, or for another microsurgical instrument used in vitrectomy, to be inserted through the trocar into the interior of the eye.

The cannula 103 of the trocar is made of a transparent ceramic. In the present illustrative embodiment, the transparent ceramic is aluminum oxynitride, although it can also be aluminum oxide or another transparent ceramic, in particular a polycrystalline transparent ceramic.

At its proximal end 113, the cannula 103 has an attachment piece for the outlet end 119 of an optical fiber, such that illumination light can be coupled via the optical fiber 121 into the transparent ceramic of the cannula 103. Outside the attachment piece 117, the entire circumferential surface of the cannula 103 is provided with a non-transparent coating 123 (see FIG. 5). Only the distal front face 125 of the cannula 103, surrounding the open distal end of the cannula 103 in a ring shape, is free of the coating 123, such that the illumination light coupled into the transparent ceramic of the cannula 103 is able to emerge from the ring-shaped front face 125 of the cannula 103. Since the front face 125 of the cannula 103 is directed toward the field of the surgical intervention with the surgical instrument guided through the interior of the cannula 103, the field of the surgical intervention can be illuminated with the aid of the cannula 103 of the trocar 101 without the need for an additional opening for a light pipe and, therefore, without the surgeon needing one hand for maneuvering a light pipe.

To permit optimal illumination of the field of the surgical intervention, the transparent material of the ring-shaped distal front face 125 in the present illustrative embodiment has a lens-shaped ground surface, which is indicated in FIG. 5 by a curved shape 127 of the ring-shaped front face 125. By means of a suitable ground surface, the light outlet direction and/or the radiation characteristics of the light on emergence from the front face 125 can thus be suitably adjusted.

A third illustrative embodiment of a surgical instrument according to the invention is described below with reference to FIGS. 6 and 7. In this third illustrative embodiment, the surgical instrument is in the form of micro forceps 201. The micro forceps 201 comprise a tube 203 through which a rod 205 is guided. At the distal end of the rod 205, two grip portions 207A, 207B are formed, which are made of a resilient material. The grip portions 207A, 207B are shaped such that the grip portions 207A, 207B are spread to the position shown in FIG. 7. By means of an actuation device 211 arranged on a handpiece 209, the bar 205 can be moved along the axial direction of the tube 203 in such a way that the grip portions 207A, 207B are partially drawn into the distal end 204 of the tube 203. A distal wall portion 213 of the tube 203 presses the grip portions 207A, 207B together counter to the pretensioning of the resilient material, such that the tips 215A, 215B of the grip portions 207A, 207B are pressed against each other and are thus able to grasp an object such as a tissue fragment or the like. The partial retraction of the grip portions 207A, 207B into the interior of the tube 203 can be obtained by pressing the actuation device 211 on the handle 209. By means of a suitable gear mechanism, the movement of the actuation device 211 generated by said pressing is converted into a movement of the bar 205 along the axial direction of the tube 203.

In the present illustrative embodiment, the tube 203 is made of aluminum oxynitride, although it can also be made of aluminum oxide or another transparent ceramic, in particular a polycrystalline transparent ceramic. At its proximal end 206, the tube 203 is provided with an attachment piece 217 for the outlet end 219 of an optical fiber 221. By means of the attachment piece 217, illumination light emerging from the outlet end 219 of the optical fiber 221 can be coupled into the transparent ceramic of which the tube 203 is made. Outside the attachment piece 217, the tube 203, except for a light outlet area 223 at the distal end 204 of the tube 203, is provided with a non-transparent coating 225, such that the light coupled into the transparent ceramic of the tube 203 can emerge again from the transparent ceramic only in the light outlet area 223. In this way, the light is guided directly into the area in which the gripping by means of the micro forceps 201 takes place.

A fourth illustrative embodiment of a surgical instrument according to the invention is shown in FIG. 8. The fourth illustrative embodiment differs from the third illustrative embodiment only in that it is in the form of micro scissors 301 instead of micro forceps 201. Elements in FIG. 8 that correspond to those from FIG. 7 are therefore designated by the same reference numbers as in FIG. 7 and, in order to avoid repetition, are not explained again.

The micro scissors 301 have in principle the same structure as the micro forceps 201 described with reference to FIG. 6 and FIG. 7, except that the grip portions 207A, 207B are replaced by blades 307A, 307B. On their sides facing toward each other, these have cutting edges 309A, 309B and, like the grip portions 207A, 207B of the micro forceps, can be pressed together counter to a pretensioning spring force by a distal wall portion 213 of a tube 203 when the blades 307A, 307B are drawn partially into the interior of the tube 203 by means of a bar which is movable along the axial direction of the tube 203 and on the distal end of which they are arranged. The mechanism is the same as has been described with reference to FIG. 7. Otherwise, the fourth illustrative embodiment does not differ from the third illustrative embodiment described with reference to FIGS. 6 and 7.

FIG. 9 shows a fifth illustrative embodiment of a surgical instrument according to the invention. The surgical instrument shown in FIG. 9 is what is called a backflush instrument, which constitutes a suction instrument for aspirating substances out of the interior of the eye.

The backflush instrument 401 comprises a suction tube 403 with an open distal end 405 and a proximal end 407. The open distal end 405 is provided for insertion into the inside of the body. At the proximal end 407 there is a handle 409 with an actuation switch 441, with which a suction device can be switched on and off. By way of a suction hose 413 connected directly or indirectly to the suction tube 403, the substance sucked in by means of the suction tube 403 is carried away when the suction device is switched on.

In the present illustrative embodiment, the suction tube 403 is made of aluminum oxynitride and has, at its proximal end 407, an attachment piece 415 for the outlet end 417 of an optical fiber 419. By means of the attachment piece 415, illumination light emerging via the outlet end 417 of the optical fiber 419 can be coupled into the transparent ceramic of the suction tube 403. Outside the attachment piece 415, the entire circumferential surface of the suction tube 403 is provided with a non-transparent coating, which prevents the illumination light from passing through the circumferential surface of the suction tube 403. At the open distal end 405, the suction tube 403 has a ring-shaped front face, which is not provided with the coating. In terms of its structure, the distal end of the suction tube corresponds substantially to the distal end of the cannula 103 of the trocar 101 as shown in FIG. 5. The only difference is that the dimensions of the suction tube 403, in particular its diameter, are typically smaller than those of the cannula 103 of the trocar 101. Like the ring-shaped front face 125 at the distal end 111 of the cannula 103 of the trocar 101, the ring-shaped front face of the distal end 405 of the suction tube 403 can also be provided with a ground surface, which ensures a desired radiation characteristic and/or a desired radiation direction of the light emerging from the front face.

For purposes of illustration, the present invention has been explained in detail on the basis of a number of illustrative embodiments. However, a person skilled in the art will appreciate that deviations from the individual illustrative embodiments are possible and that features of the individual illustrative embodiments can be combined with each other. For example, the outlet areas of the tubes of the micro forceps and of the micro scissors can also be designed like the outlet area of the cannula 103 of the trocar 101 described with reference to FIG. 5, and vice versa. Moreover, the described surgical instruments have been described with reference to ophthalmic surgery, in particular with reference to vitrectomy. However, the same instruments or similar instruments can also be used in other microsurgical interventions, for example in vascular surgery or in neurosurgery. However, surgical instruments according to the invention are also not limited to microsurgery. In principle, an embodiment of surgical instruments according to the invention may be advantageous in all minimally invasive interventions in which illumination from outside the body is difficult or indeed impossible. Therefore, the invention is not intended to be limited exclusively to the described illustrative embodiments, but only by the attached claims.

### LIST OF REFERENCE SIGNS

1 vitrector
3 cutter
5 distal end
7 proximal end
9 handpiece
11 outer tube
13 inner tube
14 open distal end
15 front wall
17 opening
18 circumferential wall
19 lumen
21 suction hose
22 space
23 attachment piece
24 outlet end
25 optical fiber
27 light outlet area
29 coating
101 trocar
103 cannula
105 lancet
107 tip
111 distal end
113 proximal end
115 instrument port
117 attachment piece
119 outlet end
121 optical fiber
123 coating
125 front face
127 ground surface
201 micro forceps
203 tube
204 distal end
205 rod
206 proximal end
207A grip portion
207B grip portion
209 handle
211 actuation device
213 distal wall portion
215A tip
215B tip
217 attachment piece
219 outlet end
221 optical fiber
223 light outlet area
225 coating
301 micro scissors
307A blade
307B blade
309A cutting edge
309B cutting edge
401 backflush instrument
403 suction tube
405 distal end
407 proximal end
409 handle
413 suction hose
415 attachment piece
417 outlet end
419 optical fiber
441 actuation switch

1. A surgical instrument in particular a microsurgical instrument, with a rod-shaped or sleeve-shaped portion that comprises a proximal end and a distal end, wherein the rod-shaped or sleeve-shaped portion is made of a transparent ceramic and has, at its proximal end an attachment piece for an optical fiber output wherein the attachment piece allows light emerging from the optical fiber output to be introduced into the transparent ceramic, and the transparent ceramic of the rod-shaped or sleeve-shaped portion is provided with a non-transparent coating except in a light outlet area.

2. The surgical instrument as claimed in claim 1, wherein the transparent ceramic is a polycrystalline ceramic.

3. The surgical instrument as claimed in claim 2, wherein the transparent ceramic is aluminum oxynitride or aluminum oxide.

4. The surgical instrument as claimed in claim 1, wherein the light outlet area is arranged closer to the distal end of the rod-shaped or sleeve-shaped portion than to the proximal end of the rod-shaped or sleeve-shaped portion.

5. The surgical instrument as claimed in claim 4, wherein the light outlet area is arranged at the distal end of the rod-shaped or sleeve-shaped portion.

6. The surgical instrument as claimed in claim 1, wherein the light outlet area has a shape that allows the light to emerge in a desired light outlet direction and/or with a desired radiation characteristic.

7. The surgical instrument as claimed in claim 1, wherein the surgical instrument is designed as a vitrector with a tubular cutter wherein the cutter comprises the rod-shaped or sleeve-shaped portion made of a transparent ceramic.

8. The surgical instrument as claimed in claim 1, wherein the surgical instrument is designed as a trocar with a cannula wherein the cannula forms the rod-shaped or sleeve-shaped portion made of a transparent ceramic.

9. The surgical instrument as claimed in claim 1, wherein the surgical instrument is designed as micro forceps in which two movable grip portions spaced apart from each other by a spring force are located at the end of a rod guided through a tube wherein the rod is movable in the longitudinal direction of the tube between a position in which the grip portions are located outside the tube and spaced apart from each other and a position in which the grip portions are located partially in the tube and are pressed together by a wall portion of the tube counter to the spring force, and in which the tube forms the rod-shaped or sleeve-shaped portion made of a transparent ceramic.

10. The surgical instrument as claimed in claim 1, wherein the surgical instrument is designed as micro scissors in which two movable blades spaced apart from each other by a spring force are located at the end of a rod guided through a tube wherein the rod is movable in the longitudinal direction of the tube between a position in which the blades are located outside the tube and spaced apart from each other and a position in which the blades are located partially in the tube and are pressed together by a wall portion of the tube counter to the spring force, and in which the tube forms the rod-shaped or sleeve-shaped portion made of a transparent ceramic.

11. The surgical instrument as claimed in claim 1, wherein the surgical instrument is designed as a suction instrument that comprises a suction tube wherein the suction tube forms the rod-shaped or sleeve-shaped portion made of a transparent ceramic.
